(19) United States
(12) Patent Application Publication
(10) Pub. No.: US 2011/0288151 A1
(43) Pub. Date: Nov. 24, 2011

(54) METHODS OF CHARACTERIZING BREAST CANCER AND IDENTIFYING TREATMENTS FOR SAME

(75) Inventor: Marcelo G. Kazanietz, Philadelphia, PA (US)

(73) Assignee: The Trustees of the University of Pennsylvania, Philadelphia, PA (US)

(21) Appl. No.: 13/122,811

(22) PCT Filed: Oct. 6, 2009
(86) PCT No.: PCT/US09/59700

§ 371 Date: Jun. 30, 2011

Related U.S. Application Data
(60) Provisional application No. 61/103,191, filed on Oct. 6, 2008.

(51) Int. Cl. A61K 31/713; C40B 30/00; A61P 35/00; C07K 16/40; A61K 31/7088; G01N 33/573; C12Q 1/68

(52) U.S. Cl. 514/44 A; 435/7.1; 506/7; 435/6.12; 530/389.8; 530/387.3; 435/6.1

(57) ABSTRACT

This application describes antibodies to P-Rex 1, and methods of detecting breast cancer, and methods of treatments for the same.

### RELATED APPLICATIONS

The present application claims the benefit of U.S. Pat. App. Ser. No. 61/103,191 (filed Oct. 6, 2008), the entirety of which application is incorporated herein by reference.

### TECHNICAL FIELD

Provided are antibodies to P-Rex1, methods of detecting breast cancer and methods of treatments for the same.

### BACKGROUND

According to the Centers for Disease Control and Prevention, breast cancer is the second most common form of cancer in women. In addition, breast cancer is the number one cause of cancer death in Hispanic women and it is the second most common cause of cancer death in white, black, Asian/Pacific Islander, and American Indian/Alaska Native women (U.S. Cancer Statistics Working Group, “United States Cancer Statistics: 2004 Incidence and Mortality,” Atlanta (GA): Department of Health and Human Services, Centers for Disease Control and Prevention, and National Cancer Institute, 2007).

Dysregulation of the human epidermal growth factor (EGF) receptor family of tyrosine kinase receptors and their effectors is common in human breast cancer, as well as other cancers. Yarden, et al., 2001, Nat. Rev. Mol. Cell. Biol., 2, 127-128. The EGF receptor family of tyrosine kinase receptors comprises 4 members: ErbB1 (EGFR or HER1), ErbB2 (Neu or HER2), ErbB3 (HER3), and ErbB4 (HER4). Id. These receptors are widely expressed in most tissues and play key roles during development and in the regulation of multiple cellular functions, including proliferation, survival, apoptosis, migration, and differentiation. Id. Members of the ErbB receptor family share considerable homology at the structural level: they all consist of an extracellular N-terminal ectodomain with binding sites for ligands of the EGF family, a transmembrane region, and an intracellular kinase domain. Id.

### HRGs: A Group of EGF-Related Peptides Involved in Human Cancer

Heregulins (HRGs), a group of EGF-like ligands identified in the early 1990's and widely expressed in tissues, are implicated in different types of cancers, particularly in breast cancer. Several HRG isoforms have been identified and classified into two groups based on their ability to bind to both ErbB3 and ErbB4 (HRG1 and HRG2) or only to ErbB4 (HRG3 and HRG4), as described in Falls, et al., 2003, Exp. Cell Res., 284, 14-30 and Stove, C., et al., 1995, J. Biol. Chem., 270, 15591-15597. HRGs are synthesized as transmembrane precursor proteins of approximately 100 kDa, which are then cleaved by metalloproteinases to soluble forms (˜45 kDa) that act in a paracrine/autocrine fashion. Intact transmembrane HRGs may also activate receptors in the same cell (juxtacrine mechanism). The HRG1 family of isoforms is the most widely studied, and among them the β isoforms, particularly in the context of mitogenesis and cancer, as described in Falls, D. L., 2003, Exp. Cell Res., 284, 14-30. The human HRG1/NRG1 gene is very long (˜1.4 mb), with several splicing sites and promoters that lead to multiple HRG1 forms. Id.

HRG is expressed in approximately 30% of breast tumors, as described in, for example, Dunn, M., et al., 2004, J. Pathol., 203, 672-680. Accumulating evidence indicates that HRG induces the progression of breast cancer cells towards an aggressive phenotype and resistance to the growth inhibitory effect of anti-estrogens in estrogen receptor (ER)-positive cells, such as MCF-7 cells, as described in Stove, C., et al., 2004, Clin. Exp. Metastatis., 21, 665-684 and Atlas, E., et al., 2003, Mol. Cancer. Res., 1, 165-175. HRG triggers indirectly the activation of ErbB2; ErbB2/ErbB3 and ErbB2/ErbB4 dimers can be detected upon HRG stimulation, as described in, for example, Citri, A., et al., 2006, Nat. Rev. Mol. Cell. Biol., 7, 505-516 and Citri, A. et al., 2003, Exp. Cell Res., 284, 54-65. ErbB2 is the preferred dimerization partner for ErbB3 and ErbB2/ErbB3 dimers have remarkable signaling potency due to a slow rate of ligand dissociation and evasion of endocytosis, as described in, for example, Yarden, Y., 2001, Oncology, 61, Suppl. 2, 1-13. HRG signals for the activation of ERK, PI3K, and PLCγ pathways, leading to important mitogenic and survival responses, cyclin D1 induction, activation of CDKs, and Rb phosphorylation. HRG-induced proliferation and transformation are inhibited by functionally blocking ErbB2, as described in Aigner, A., et al., 2001, Oncogene, 20, 2101-2111. Notably, the population of breast tumors with HRG overexpression is distinct from those overexpressing ErbB2, and HRG is a potent mitogen in cells with low ErbB2 levels, as described in Menendez, J. A., J. Clin. Oncol., 24, 3735-3746. HRG promotes migration and the formation of ruffles, filopodia, and stress fibers, as described in Adam, L., et al., 1998, J. Biol. Chem., 273, 28238-28246, suggesting that it activates Rho GTPases.

### Rac and its Involvement in Breast Cancer

HRG is an activator of Rac in breast cancer cells, as described in Yang, et al., 2006, Mol. Cell. Biol., 26, 831-842. Rac is also activated by EGF. Id. Furthermore, HRG-induced activation of Rac is dependent on EGFR. Id. Importantly, HRG-induced proliferation is dependent on Rac. Id.

Rac belongs to the family of Rho small GTPases that relay signals from tyrosine kinases and GPCRs. Of more than 20 members of the Rho GTPases, Rac1, Cdc42 and RhoA have been the best characterized, as described in, for example, Etienne-Manneville, S., et al., 2002, Nature, 420, 629-635. Early studies revealed that Rac plays a key role in actin cytoskeleton reorganization, as described in Hall, A., 1998, Science, 279, 509-514. Subsequent studies revealed that Rac is also an important regulator of migration, adhesion, cell cycle progression, gene expression, transformation, and metastasis, as discussed in, for example, Jaffe, A. B., et al., 2005, Annu. Rev. Cell Dev. Biol., 21, 247-269. Three Rac isoforms have been identified: Rac1, Rac2 and Rac3. While Rac2 is exclusively expressed in hematopoietic cells and Rac3 is mainly expressed in brain, Rac1 is ubiquitously expressed, as described in Takai, Y., et al., 2001, Physiol. Rev., 81, 153-208. A Rac1 splice variant (Rac1b) has also been identified, as described in Jordan, P., et al., 1999, Oncogene, 18, 6835-6839. Rac and other Rho GTPases can be overexpressed in cancer, including in breast cancer, as discussed in, for example, Jordan, P., et al., 1999, Oncogene, 18, 6835-6839.

Like most other small GTPases, Rac cycles between an active GTP-bound state and an inactive GDP-bound state. Rac-GTP interacts with a number of downstream effectors, including Pak1 and IRSp53, as described in, for example, Miki, H., et al., 2000, Nature, 408, 732-735. The activity of Rac is mainly regulated by three classes of proteins: i) guanine nucleotide exchange factors (GEFs), which promote the exchange of GDP to GTP via Dbl domains to activate Rac (for example, Tiam-1 and Vav isoforms); ii) guanine nucleotide dissociation inhibitors (GDIs), which limit the access of the GTPase to GEFs and other regulators, keeping the G-protein in the GDP-bound state; and iii) GTPase activating proteins (GAPS), which accelerate the intrinsic GTPase activity of Rac, thus leading to its inactivation (for example, α- and β-chimaerins and 3BP-1). Cellular signaling from tyrosine kinase receptors to Rac is mainly mediated by their direct or indirect coupling to GEFs. Tiam1 and other Rac-GEFs have PH domains that bind to PI3K products and regulate their membrane association, as described in, for example, Fleming, I. N., et al., 2004, Biochem. J., 382, 857-865. GEFs may also associate directly to tyrosine kinase receptors, as described for Vav with EGFR, as described in Tamas, P., et al., 2003, J. Biol. Chem., 278, 5163-5171, or they may become activated in a PI3K-dependent manner, as described in Baumeister, M. A., et al., 2005, J. Biol. Chem., 278, 11457-11464. Signals from diverse tyrosine kinase receptors can converge on the same Rho-GEF. Alternatively, a single tyrosine kinase receptor can activate multiple GEFs. Rho-GEFs can be promiscuous in terms of small G-protein activation (for example, Vav2 activates Rho and Rac) or possess remarkable selectivity, as indicated by the fact that Tiam1 is the only GEF associated with Rac in certain cell types. Minard, et al., 2004, Breast Cancer Res. Treat., 84, 21-32.

There is evidence for hyperactivation of the Rac pathway in human cancer, including in breast cancer. Rac-GAPS have been reported to be down-regulated in human breast tumors, as described in Yang, C., et al., 2005, J. Biol. Chem., 280, 24363-24370. Rac itself is overexpressed in human cancer, including in breast cancer, as described in Schnelzer A., et al., 2000, Oncogene, 19, 3013-3020. Overexpression of Rac-GEFs, such as Tiam1, contributes to cancer progression and metastatic dissemination, as described in Minard, G. E., et al., 2004, Breast Cancer Res. Treat., 84, 21-32. Many inputs in breast cancer can potentially signal to Rac and result in Rac hyperactivation, such as PTEN mutation (high PI3K activity), mutations in the PI3KCA gene or ErbB2 overexpression. There is also strong evidence that Pak1, a direct Rac effector, is hyperactive in human breast tumors, which correlates with cyclin D1 overexpression and tumor grade, as described in Blasenthil, S., et al., 2004, J. Biol. Chem. 279, 1422-1428 and Salh, B., et al., 2002, Int. J. Cancer 98, 148-154.

Emerging evidence has linked Rac to estrogen-dependence and anti-estrogen resistance. For example, overexpression of the Rac activator AND-34/BCAR3 promotes anti-estrogen resistance via cyclin D1 induction, as described in Near., R. I., et al., 2007, J. Cell. Physiol., 212, 655-665, and Pak1 hyperactivation correlates with tamoxifen resistance in breast cancer patients, as described in Holm, C., et al., 2006, J. Natl. Cancer Inst., 98, 671-680.

Further elucidating the molecular signaling pathways involving HRG, EGF, and Rac can help identify targets of therapy for breast cancer. Additional signaling involving Rac have been described in Yang, et al., 2005, 280, 24363-24370; Yang, et al., 2006, Mol. Cell. Biol., 26, 831-842; and Yang, et al., 2008, Biochem, J. 410, 167-175, each of which is incorporated by reference herein.

### P-Rex1 and Rac in Breast Cancer Cells

Identification of upstream or downstream modulators of Rac would provide additional targets of therapy for breast cancer. P-Rex1, phosphatidylinositol-3,4,5-triphosphate-dependent Rac Exchanger-1 or phosphatidylinositol-3,4,5-triphosphate-dependent Rac Exchange Factor-1, is a PI3K and Gβγ-regulated Rac-GEF originally identified in neutrophils, as described in Welch, et al., 2002, Cell, 108, 809-821. Herein, P-Rex1 is shown, among other things, to activate Rac in breast cancer cells.

### SUMMARY

Described herein are methods of detecting cancer in breast cell or tissue samples wherein the levels of P-Rex1 expression in the samples is measured and compared. In one embodiment, the method of detecting cancer in a sample of breast tissue comprises measuring the level of expression of P-Rex1 in the sample and comparing that level with the level of expression of P-Rex1 in a control sample. In an embodiment of this method, the mammal is a human.

In an embodiment of this method, the control sample would be non-cancerous tissue. In another embodiment of this method, the control sample would be a sample known to contain no P-Rex1. In another embodiment of this method, the control sample would be a sample known to contain low levels of P-Rex1.

In an embodiment of this method, the comparing step comprises comparing the levels of expression of P-Rex1 mRNA. In a further embodiment of this method, the levels of expression of P-Rex1 mRNA are measured by quantitative PCR.

In an embodiment of this method, the comparing step comprises comparing the levels of expression of P-Rex1 protein. In a further embodiment of this method, the levels of P-Rex1 protein are measured using an antibody to P-Rex1. In a further embodiment, the antibody used to measure P-Rex1 protein is derived from an animal presented with a peptide comprising the amino acid sequence of SEQ ID NO:1 (QEEDQADSAFPLLSLGPRLSLC). In a further embodiment, the antibody used to measure P-Rex1 protein is derived from an animal presented with a peptide consisting of the amino acid sequence of SEQ ID NO:1.

Described herein are antibodies that detect P-Rex1 protein. In an embodiment of the antibody, the antibody is derived from an animal presented with a peptide comprising the amino acid sequence of SEQ ID NO:1 (QEEDQADSAFPLLSLGPRLSLC). In another embodiment, the antibody is derived from an animal presented with a peptide consisting of the amino acid sequence of SEQ ID NO:1. In further embodiments, any derived antibody is in humanized form.

Described herein are methods of determining P-Rex1 expression in a sample. In an embodiment of the method, the method comprises contacting a sample with an antibody derived from an animal which has been presented with a peptide comprising the amino acid sequence of SEQ ID NO:1 and detecting binding of the antibody with at least some, if any, P-Rex1 present in the sample. In another embodiment of the method, the method comprises contacting a sample with an antibody derived from an animal which has been presented with a peptide consisting of the amino acid sequence of SEQ ID NO:1 and detecting binding of the antibody with at least some, if any, P-Rex1 present in the sample.

Described herein are methods of identifying a compound useful for treating breast cancer. An embodiment of the method comprises contacting a compound with breast cancer cells and comparing the expression of P-Rex1 in the cells contacted with the compound with the expression of P-Rex1 in similar cells not contacted with the compound, wherein a compound that reduces the level of expression of P-Rex1 is determined to have such utility. In another embodiment, a method of identifying a compound useful for treating breast cancer comprising contacting the compound with breast cancer cells and comparing the activity of P-Rex1 in the cells contacted with the compound with the activity of P-Rex1 in similar cells not contacted with the compound, wherein a compound that reduces the level of expression of P-Rex1 is determined to have such utility.

In some embodiments, the compound is a nucleic acid construct. In other embodiments, the compound is a nucleic acid comprising an shRNA, siRNA, or antisense construct, or a combination thereof.

Described herein is a method of inhibiting breast cancer growth, progression, or metastasis, or a combination thereof, comprising inhibiting P-Rex1 activity or expression, or both. Also described herein is a method of treating breast cancer comprising inhibiting P-Rex1 activity or expression, or both.

In some embodiments, inhibiting P-Rex1 activity or expression, or both, comprises administering a nucleic acid comprising an shRNA, siRNA, or antisense construct, or a combination thereof.

In some embodiments, the breast cancer is estrogen receptor negative or HER2 negative, or both. In other embodiments, the breast cancer is estrogen receptor positive or HER2 positive or both. In other embodiments, the breast cancer is estrogen receptor negative or HER2 positive, or both. In other embodiments, the breast cancer is estrogen receptor positive or HER2 negative, or both.

### BRIEF DESCRIPTION OF THE DRAWINGS

FIG. 1 is an image of an immunoblot depicting Rac-GTP levels in various breast cancer cell lines treated with HRG (10 ng/mL) or EGF (100 ng/mL) for 0-5 minutes, as detected by an anti-Rac antibody following a GST-Raf-PBD pull-down assay of lysates of the treated cells.

FIG. 2 is an image of an immunoblot depicting Rac activation as measured by Rac-GTP levels in human MDA-MB-231 tumor cells grown as xenografts in nude mice, after treatment with and without an HRG antisense construct, as detected by an anti-Rac antibody following a GST-Rac-PBD pull-down assay of lysates of the tumor cells.

FIG. 3 is a graph plotting relative expression of mRNA levels of 26 Rac-GEFs or Rac-GEF regulators (P-Rex1 (PR), VAV3 (V3), ARHGEF2 (A2), BCAR3 (B3), ARHGEF7 (A7), SWAP70 (SW), TRIO (TR), SOS1 (S1), VAV2 (V2), SOS2 (S2), ELMOD2 (E2), DEF6 (D6), VAV1 (V1), DOCK1 (D1), FARP2 (F2), ARHGEF6 (A6), ALS2 (A2), Tiam1 (T1), GEFT (GE), Tiam2 (T2), RasGRF1 (R1), KALRN (Ka), RasGRF2 (R2), DEPDC2/P-Rex2 (D2), ELMOD1 (E1), and MCF2 (M2) (two letter abbreviation used in FIG. 3 is shown in parentheses following the name of the gene)) in T-47D and MCF-7 breast cancer cell lines as determined by real-time PCR. Each sample was assayed in triplicate in each experiment. Relative expression of each gene is shown as a percentage of the level of P-Rex1.

FIG. 4A is a image of an immunoblot and an electrophoretic gel. The immunoblot image depicts Rac-GTP levels in T-47D breast cancer cells transfected with RNAi duplexes (SMARTpool, Dharmacon) targeting ARHGEF2, ARHGEF7, BCAR3, ELMOD2, PREX1, SOS1, SOS2, SWAP70, Tiam1, TRIO, VAV2, and VAV3, subjected to 48 hour incubation, serum starved for 24 hours, and treated for 5 minutes with HRG (10 ng/mL). Rac-GTP levels were determined by a Rac-GTP pull-down assay of lysates of the treated cells. The image of the electrophoretic gel shows the levels of expression of P-Rex1 and GAPDH as detected by resolution of the respective RT-PCR products.

FIG. 4B is an image of a typical immunoblot depicting P-Rex1 expression in control cells and cells transfected with P-Rex1 RNAi using an antibody derived from a rabbit presented with a peptide consisting of the amino acid sequence of SEQ ID NO:1.

FIG. 4C is an image of an immunoblot depicting Rac-GTP levels in T-47D breast cancer cells transfected with P-Rex1 RNAi followed by a 48 hour incubation, serum starvation for 24 hours, and treatment for 1 minute with EGF (100 ng/mL). Rac-GTP levels were determined by a Rac-GTP pull-down assay of lysates of the treated cells. Total Rac is shown as a loading control.

FIG. 5 is an image of an immunoblot depicting Rac-GTP levels in MCF-7 breast cancer cells transfected with P-Rex1 RNAi followed by a 48 hour incubation, serum starvation for 24 hours, and treatment for 5 minutes with HRG (10 ng/mL). Rac-GTP levels were determined by a Rac-GTP pull-down assay of lysates of the treated cells.

FIG. 6 is an image of an immunoblot depicting Rac-GTP and Phospho-Akt levels in T-47D breast cancer cells transfected with P-Rex1 RNAi followed by a 48 hour incubation, serum starvation for 24 hours, and treatment for 5 minutes with HRG (10 ng/mL). Rac-GTP levels were determined by a Rac-GTP pull-down assay of lysates of the treated cells. Phospho-Akt levels were also determined by immunoblotting. Total Rac is shown as a loading control.

FIG. 7A is an image of an immunoblot depicting P-Rex1 expression, as detected by an antibody derived from a rabbit presented with a peptide consisting of the amino acid sequence of SEQ ID NO:1., in T-47D breast cancer cells transfected with four different P-Rex1 RNAi duplexes (P1, P2, P3, P4) followed by incubation for 48 hours, serum starvation for 24 hours, and treatment for 5 minutes with HRG (10 ng/mL). Actin is shown as a loading control.

FIG. 7B is an image of an immunoblot depicting Rac-GTP levels in T-47D breast cancer cells transfected with one four P-Rex1 RNAi duplexes (P1, P2, P3, P4) or a Vav3 RNAi duplex, followed by a 48 hour incubation, serum starvation for 24 hours, and treatment for 1 minute with EGF (100 ng/mL). Rac-GTP levels were determined by a Rac-GTP pull-down assay of lysates of the treated cells. Total Rac is shown as a loading control.

FIG. 8 is an image of an ethidium bromide stained agarose gel depicting the lack of P-Rex2 (DEPCC2) expression in T-47D and MCF-7 breast cancer cells, as determined by electrophoresis of the product of an RT-PCR assay performed on lysates of the cells.

FIG. 9A is a bar graph plotting the relative expression of mRNA of P-Rex1 in 6 different cell lines, MCF-10A, MCF-10AT, MDA-MB-468, MDA-MB-453, T-47D, and MCF-7. Relative expression is displayed as mRNA copy number normalized to mRNA copy number of 18S in the same sample.

FIG. 9B is a bar graph plotting the relative expression similarly to that shown in FIG. 9A, including additional relative expression of mRNA of P-Rex1 in BT-474 cells, which normally express very high P-Rex1 levels, over-express ErbB2/HER2, a common genetic alteration in breast cancer, and depend on ErbB2/HER2 for their ability to develop tumors in nude mice.

FIG. 10 is a series of graphs depicting normalized expression of P-Rex-1 (top graph), P-Rex1 normalized to cytokeratin 10 (middle graph), and myeloperoxidase (bottom graph) in normal breast tissue (Stage 0) and Stage I, IIa, IIb, IIIa, IIIc, and IV breast cancer samples, as measured by quantitative PCR of cDNA prepared from the samples (Origene TissueScan cDNA panel). In the data in each graph, mRNA copy number for each gene is first normalized to actin mRNA.

FIG. 11A is a typical image of immunohistochemical staining of P-Rex1 in tumor cells in two human breast tumor samples using a 1:100 dilution of an antibody derived from a rabbit presented with a peptide consisting of the amino acid sequence of SEQ ID NO:1.

FIG. 11B is a summary of the stained samples exemplified in FIG. 11A.

FIG. 12A is an image of immunohistochemical staining of P-Rex1 in MCF-7 breast cancer cells that were serum starved for 48 hours, treated or not treated for 5 minutes with HRG (10 ng/mL), fixed, and stained with an antibody derived from a rabbit presented with a peptide consisting of the amino acid sequence of SEQ ID NO:1.

FIG. 12B is an image of immunohistochemical staining of P-Rex1 in MCF-7 breast cancer cells that were serum starved for 48 hours, treated or not treated for 10 minutes with HRG (10 ng/mL), treated or not treated with wortmanin, control IgG, or anti-Erb3 antibody, and then fixed, and stained with an antibody derived from a rabbit presented with a peptide consisting of the amino acid sequence of SEQ ID NO:1.

FIG. 13A is an image of an immunoblot depicting the lack of P-Rex1 protein expression in T-47D cells transfected with 2 different lentiviruses (L#1, L#2) that each encode shRNA targeting P-Rex1 (Sigma).

FIG. 13B is a bar graph plotting the normalized expression of 14 Rac-GEFs (P-Rex1 (PR), VAV3 (V3), BCAR3 (B3), ARHGEF2 (A2), SWAP70 (SW), ARHGEF7 (A7), SOS1 (S1), VAV2 (V2), ELMOD2 (E2), TRIO (TR), SOS2 (S2), DOCK1 (D1), PARP2 (P2), and VAV1 (V1)) in T-47D breast cancer cells transfected with the 2 different lentiviruses (L#1, L#2) described in FIG. 13A. Expression is normalized to GAPDH.

FIG. 14A is an image of an immunoblot depicting the lack of P-Rex1 protein expression in T-47D cells transfected with 4 different pools (93, 94, 96, 97) of lentiviruses that each encode shRNA targeting P-Rex1 (Sigma) and selected using puromycin.

FIG. 14B is an image of an immunoblot depicting Rac activation, in response to treatment with HRG (10 ng/mL), in the P-Rex1 depleted cell lines of FIG. 14A.

FIG. 14C is a bar graph plotting the percent migration, in response to treatment with HRG (10 ng/mL), of the P-Rex1 depleted cell lines of FIG. 14A.

FIG. 14D is a bar graph depicting the percentage of cells, wherein the cells are the P-Rex1 depleted cell lines of FIG. 14A, exhibiting ruffles in response to treatment with HRG (10 ng/mL).

FIG. 14E is a bar graph depicting colony formation, in response to treatment with HRG (10 ng/mL), in the P-Rex1 depleted cell lines of FIG. 14A.

FIG. 15A is an image of an immunoblot depicting typical results Rac activation in MCF-7 or T-47D cells that were serum starved for 24 hours, followed by addition of pertussis toxin (PTX; 100 ng/mL) for 24 hours, and treatment with HRG (10 ng/mL) in the presence of serum.

FIG. 15B is a bar graph depicting percent Rac activation of the cells described in FIG. 15A.

FIG. 15C is an image of an immunoblot depicting Rac activation in MCF-7 or T-47D cells that were serum starved for 24 hours, followed by addition of pertussis toxin (PTX; 100 ng/mL) for 24 hours, and treatment with EGF (100 ng/mL) in the presence of serum.

FIG. 16A is an image of an immunoblot depicting that the PI3Kγ inhibitor 5-quinoxalin-6-ylmethylene-thiazolidine-2,4-dione reduces Rac activation by HRG (100 ng/ml, 5 min) in MCF-7 cells.

FIG. 16B is an image of an immunoblot depicting MCF-7 cell lines that were generated to target PI3Kγ expression (#1 and #2), including a non-target sequence (NTS) control, using lentiviruses that result in expression of shRNAs.

FIG. 16C is an image of an immunoblot depicting that PI3Kγ depletion, in the MCF-7 cell lines of FIG. 16B, impairs Rac activation by HRG.

FIG. 16D is a bar graph depicting percent Rac activation of the cells described in FIGS. 16B and 16C.

FIG. 17 is an image of an immunoblot depicting Rac activation in serum-starved MCF-10A cells treated with HRG (100 ng/ml, 5 min), with or without pretreatment with PI3K inhibitor wortmannin, EGFR inhibitor AG1478, PI3Kγ inhibitor 5-quinoxalin-6-ylmethylene-thiazolidine-2,4-dione (1 μM, 1 h), or PTX (24 h) (n=3).

FIG. 18A is an image of an immunoblot depicting Rac activation by SDF-1α in MCF-7 cells.

FIG. 18B is a bar graph plotting the percent migration of the P-Rex1 depleted cell lines of FIG. 18A in response to SDF1-α.

FIG. 18C is an image of an immunoblot depicting Rac1 activation in T-47D cells subject to P-Rex1 depletion using shRNA lentiviruses (L#2) or control shRNA lentivirus (C).

FIG. 18D is a bar graph plotting the percent migration of the P-Rex1 depleted cell lines of FIG. 18C in response to SDF1-α (10 ng/mL).

FIG. 18E is an image of an immunoblot depicting Rac activation in response to HRG (100 ng/mL, 5 min.) in MCF-7 cells 48 hours after transfection with CXCR4 or control (C) siRNA duplexes

FIG. 18F is a bar graph plotting the percent migration of the CXCR4 depleted cell lines of FIG. 18E in response to HRG.

FIG. 19 is a schematic depicting ErbB receptor signaling through P-Rex1 and Rac.

FIG. 20A is graph plotting tumor area versus number of days, and depicting inhibition of tumor formation with of P-Rex1-depleted BT-474 cell lines as compared to control cells, wherein each was injected into the flanks of athymic (nude) female ovariectomized mice (2×10⁷ cells/mouse).

FIG. 20B is bar graph depicting decreased levels of Rac activation, as compared to control, in the P-Rex1 depleted BT-474 cell lines used in the experiment shown in FIG. 20A.

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

A “culture of cells” or “cell culture” or “cultured cells” refers to multiple methods that are well known in the art to propagate various cell lines, or cells derived from tumors in the laboratory. Methods for doing so are well known in the art, including the cell culture methods described herein. Unless otherwise stated, substitution of other methods for culturing is common in the art and within the scope of the disclosed subject matter herein.

A cell has been “transformed” or “transfected” by exogenous or heterologous nucleic acids such as DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell, or “stable cell” is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts, monographs, and research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural posttranslational processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., Analysis for Protein Modifications and Nonprotein Cofactors, Meth Enzymol (1990) 182:626-646 and Rattan et al., Protein Synthesis: Posttranslational Modifications and Aging, Ann NY Acad Sci (1992) 663:48-62).

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression/production of an antibody or antigen-binding fragment can be within the cytoplasm of the cell, and/or into the extracellular milieu such as the growth medium of a cell culture.

As used herein, “breast cancer” refers to any stage of abnormal growth or migration of breast cells or cells that derive from breast tissue, including precancerous and all stages of cancerous cells, including but not limited to metaplasias, heteroplasias, dysplasias, neoplasias, hyperplasias, and anaplasias.

As used herein, “breast cancer progression” refers to any measure of cancer growth, development, and/or maturation including metastasis. “Breast cancer progression” includes increase in cell number, cell size, tumor size, and number of tumors, as well as morphological and other cellular and molecular changes and other staging characteristics.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

“Antibody” refers to all isotypes of immunoglobulins (IgG, IgA, IgE, IgM, IgD, and IgY) including various monomeric and polymeric forms of each isotype, and functional fragments thereof, such as an Fab or CDR fragment that is able to detect its antigen over background/non-specific binding, unless otherwise specified. Methods for humanizing antibodies or functional fragments thereof are known in the art.

As used herein, “P-Rex1 activity” or the “activity of P-Rex1” refers to both direct or indirect activity associated with P-Rex. For example, as P-Rex1 is a guanidine exchange factor expressed intracellularly, “P-Rex1 activity” can refer to the enzymatic action of P-Rex1. In addition, as is known in the art and disclosed herein, P-Rex1 binds to and activates Rac. Accordingly, “P-Rex1 activity” includes activation and/or binding of P-Rex-1 to Rac. Assays for measuring the activity of P-Rex1 are known in the art and/or disclosed herein.

As used herein, “RNAi duplexes” refer to synthetic nucleic acid constructs that result in reduction or elimination of gene expression through the phenomenon of RNA interference, which is known in the art. Synthetic antisense oligonucleotides also known in the art to reduce or eliminate gene expression and are used herein.

As used herein, “Nucleic acid constructs” refer to any nucleic acid including DNA or RNA, double stranded or single stranded or in a vector, synthetic or natural, with or without regulatory sequences such as promoters or enhancers, and with or without modified bases or other chemical modifications. Nucleic acid constructs may be in a variety of forms, such as single stranded or double stranded oligonucleotides, plasmids, or viral vectors, such as lentiviral vectors, and the form of the construct will be evident from context as understood by one of skill in the art.

Nucleic acid constructs that induce the phenomenon of RNA interference when introduced into cells are known in the art, and include synthetic short hairpin RNA (shRNA) and synthetic short interfering RNA (siRNA), which are also known in the art. Antisense constructs are known in the art as is their design to knock-down expression of a gene. Not wishing to be bound by any particular theory, knockdown of gene expression can be achieved with the use of antisense oligonucleotides that rely on ribonuclease H(RNAse H)-dependent cleavage of mRNA, and RNA interference triggered by small double-stranded RNA molecules. Both methods act in a sequence-specific manner and can give efficient knockdown. In both cases, testing and selection of nucleic acid constructs for knockdown of gene expression can be performed to reduce or eliminate nonspecific off-target effects; such testing and selection is known to one of skill in the art.

It is to be understood that the embodiments described herein are not limited to particular methods, reagents, compounds, compositions or biological systems, which can, of course, vary. It is also to be understood that the terminology used herein is for the purpose of describing: particular antibodies or antigen-binding fragments; detection of cancer; comparing expression levels of mRNA or protein (such as that of P-Rex1); detecting expression of a gene (such as P-Rex1); and identifying compounds useful for treating cancer only, and is not intended to be limiting.

### EXAMPLES

### Example 1

### Antibody to P-Rex1

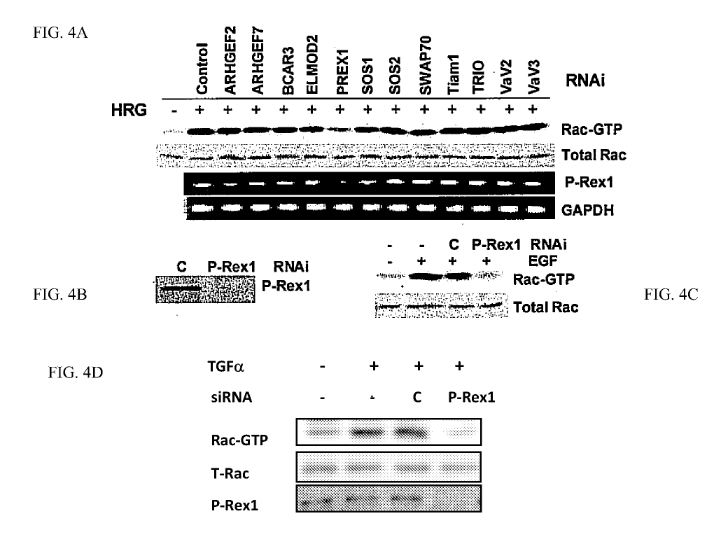

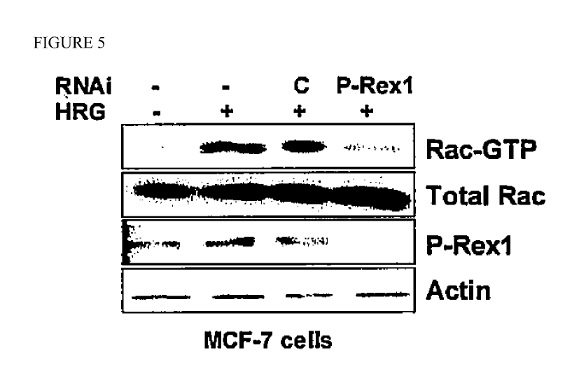

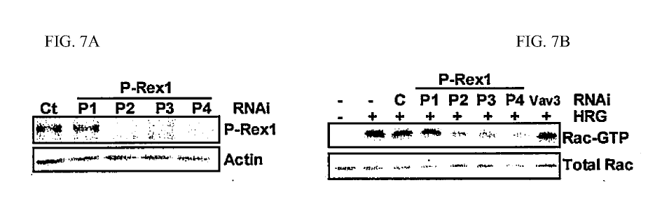

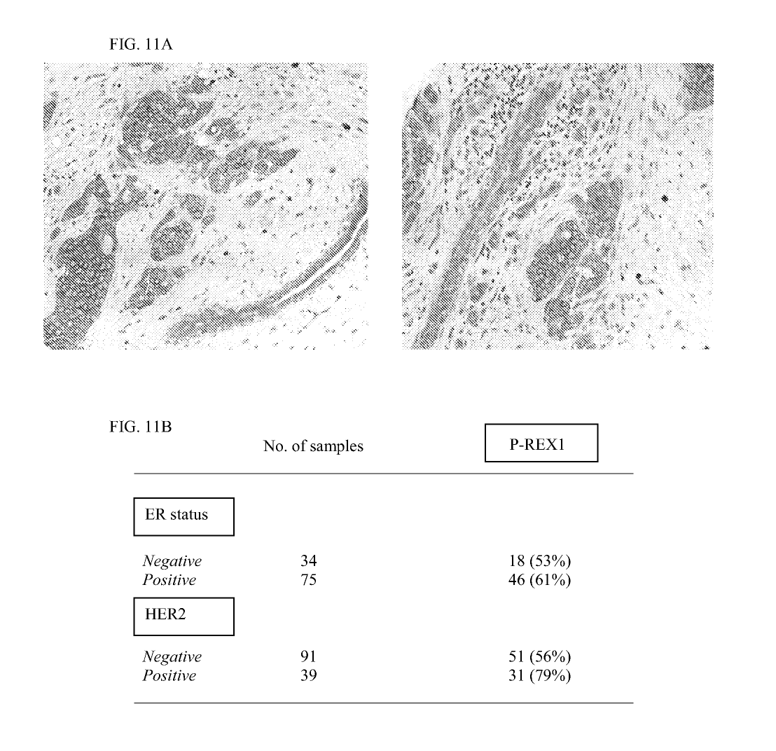

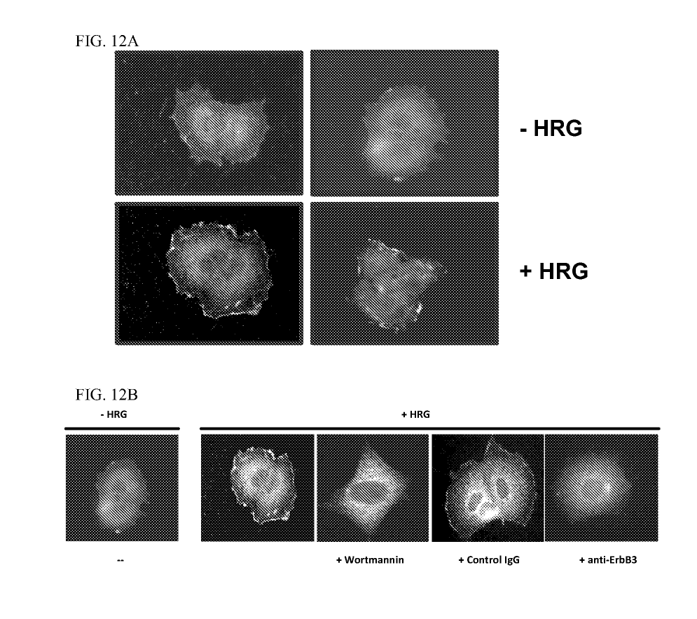

At least one other antibody generated against P-Rex1 exhibited non-specificity, as indicated by non-specific staining in immunohistochemistry (data not shown). Thus, an antibody to P-Rex1 was generated by presenting rabbits with an adjuvant comprising a 22 amino acid peptide: QEEDQADSAFPLLSLGPRLSLC (SEQ ID NO: 1). This antibody exhibits specificity to P-Rex1 in immunoblotting (examples shown throughout including FIG. 4B, FIG. 5, and FIG. 7A) and immunohistochemistry (examples shown throughout including FIGS. 11A, 11B, 12A, and 12B).

### Example 2

### HRG and EGF Treatment of Breast Cancer Cells

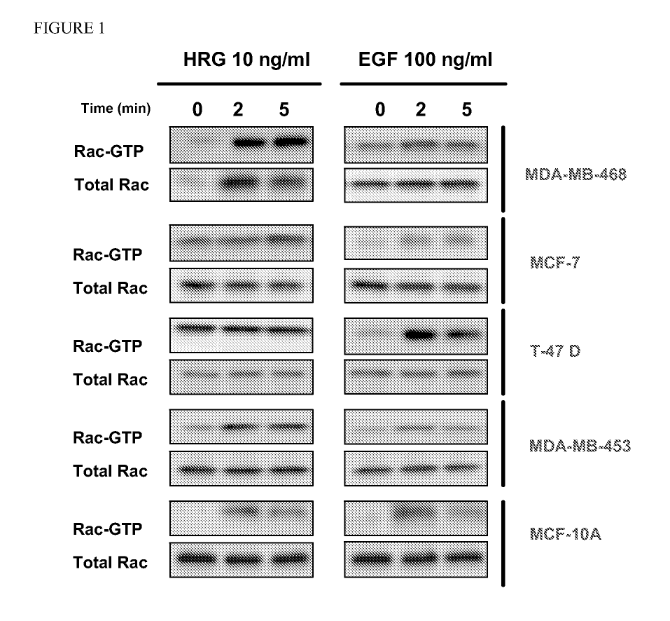

To determine the kinetics of Rac activation in breast cancer cells, the various cancer cell lines were grown in culture, serum starved for 48 hours, and stimulated with HRG (10 ng/mL) or EGF (100 ng/mL for 0-5 minutes and subject to a GST-Raf-PBD pull-down assay. The levels of activated Rac were then assessed by immunoblotting with an antibody (FIG. 1). As shown in FIG. 1, HRG and EGF activate Rac in various breast cancer cell lines (n=3).

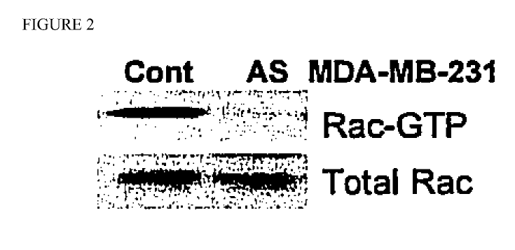

Rac hyperactivation in response to HRG treatment was also observed in human MDA-MB-231 cells as shown in FIG. 2. These cells were expanded as xenografts in nude mice, excised, and then frozen; a portion were subject to HRG antisense and exhibited slow growth as xenografts. Activated Rac levels were measured by a modified PBD pull down assay and immunoblotting. Tumor cells subject to antisense targeting HRG did not exhibit high levels of activated Rac.

### Example 3

### P-Rex1 is Highly Expressed in Breast Cancer Cells

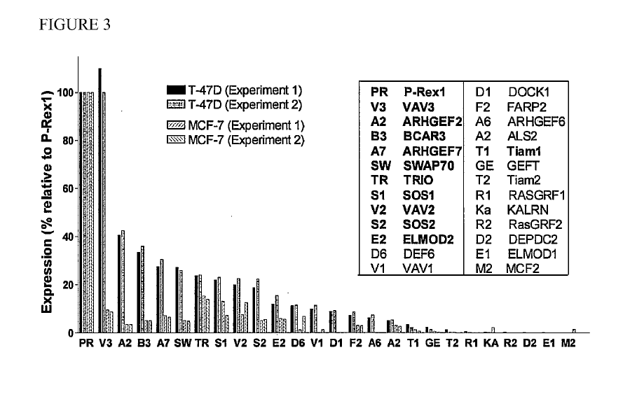

To determine which Rac-GEFs mediated HRG-induced Rac activation, a custom PCR array was designed (SuperArray Biosystems). It permitted simultaneous assessment of the mRNA expression of 26 Rac-GEFs, namely (P-Rex1 (PR), VAV3 (V3), ARHGEF2 (A2), BCAR3 (B3), ARHGEF7 (A7), SWAP70 (SW), TRIO (TR), SOS1 (S1), VAV2 (V2), SOS2 (S2), ELMOD2 (E2), DEF6 (D6), VAV1 (V1), DOCK1 (D1), FARP2 (F2), ARHGEF6 (A6), ALS2 (A2), Tiam1 (T1), GEFT (GE), Tiam2 (T2), RasGRF1 (R1), KALRN (Ka), RasGRF2 (R2), DEPDC2/P-Rex2 (D2), ELMOD1 (E1), and MCF2 (M2) (two letter abbreviation used in FIG. 3 is shown in parentheses following the name of the gene)) in addition to 4 housekeeping genes and 2 negative controls.

To perform the array experiments, RNA was isolated from T-47D cells and MCF-7 cells and reverse transcribed. The resulting cDNA was used to perform real time PCR (ABI 7300) using the PCR primers and reagents provided with the custom PCR array. Two independent cultures of each of T-47D and MCF-7 cells were used to perform the experiment, and each sample in each experiment was performed in triplicate. In FIG. 3, the expression of each gene is shown as a percentage of that of P-Rex1; P-Rex1 exhibited the highest level of expression in both cell lines. In contrast, the non-transformed but immortalized breast cell line MCF-10A did not express high levels of P-Rex1 (data not shown).

### Example 4

### P-Rex1 Mediates Rac Activation by ErbB Ligands which is Specifically Blocked by P-Rex1 Depletion by RNAi

To determine which Rac-GEF, was associated with Rac in T-47D and MCF-7 breast cancer cells, RNAi experiments targeting the expression of P-Rex1 were performed (FIGS. 4, 5, 6, and 7).

T-47D breast cancer cells were subject to RNAi for one of 12 Rac-GEFs, namely ARHGEF2, ARHGEF7, BCAR3, ELMOD2, PREX1, SOS1, SOS2, SWAP70, Tiam1, TRIO, VAV2, and VAV3, or a control RNAi duplex (SMARTpool, Dharmacon). After incubation for 48 hours, the cells were serum starved and either left untreated as a control sample, or treated with HRG (10 ng/mL) for 5 minutes. Cell lysates were then subject to a PBD pull-down assay to measure activated Rac. The top panel of FIG. 4A shows the results of the PBD assay as detected by immunoblotting with an anti-Rac-GTP antibody and an anti-Rac antibody.

The electrophoretic gel in the bottom panel of FIG. 4A shows that the level of expression of P-Rex1 was only affected by the specific nucleic acid construct designed to target P-Rex1, and that inhibition of expression of P-Rex1 also inhibits activation of Rac in response to HRG treatment.

Significantly, the T-47D breast cancer cells subject to RNAi for P-Rex1 exhibited lower levels of activated Rac, specifically a greater than 80% reduction of Rac-GTP relative to the other HRG treated cells (either subject to HRG treatment or RNAi targeting one of the other 11 Rac-GEFs followed by HRG treatment) (FIG. 4A). To verify that the P-Rex1 RNAi construct was specific, RT-PCR was performed using primers for P-Rex1 and GAPDH, and the products were separated by size in an agarose gel via electrophoresis and stained with ethidium bromide. The images of the gel show the bands corresponding to the expected size for P-Rex1 and GAPDH, and verify that the P-Rex1 RNAi was effective at inhibiting P-Rex1 mRNA expression (FIG. 4A).

The efficacy of the P-Rex1 RNAi constructs was also verified by testing the expression of P-Rex1 protein by immunoblotting post-RNAi treatment. Four P-Rex1 RNAi duplexes (P1, P2, P3, and P4) were tested for their ability to block P-Rex1 expression in T-47D cells. After transfection, three of the four constructs (P2, P3, and P4) caused a greater than 90% reduction of P-Rex1 protein as measured by a Western blot using the antibody of Example 1 (FIG. 7A). Typical results are shown in FIG. 4B and FIG. 7B.

Only those constructs which inhibited P-Rex1 protein expression, namely P2, P3, and P4, blocked activation of Rac by HRG, as shown by a PBD pull-down assay; P1 did not block activation of Rac by HRG (FIG. 7B). Furthermore, Vav3 RNAi and a control RNAi construct did not block activation of Rac by HRG (FIG. 7B), demonstrating the efficacy of the constructs designed and used for these RNAi experiments.

MCF-7 breast cancer cells subject to RNAi targeting P-Rex1 behaved similarly to T-47D breast cancer cells; P-Rex1 RNAi treatment followed by HRG stimulation blocked substantial activation of Rac in MCF-7 breast cancer cells as shown in FIG. 5.

Since EGF treatment, like HRG treatment, activates Rac as shown in FIG. 1, the involvement of P-Rex1 in EGF-stimulated activation of Rac was tested. As shown in FIG. 4C, cells subject to treatment with RNAi targeting P-Rex1 followed by EGF treatment (100 ng/mL, 1 minute) also showed greatly reduced levels of activated Rac.

Finally, TGFα-induced activation of Rac was also inhibited by P-Rex1 depletion as shown in FIG. 4D.

### Example 5

### HRG Activation of Rac is P-Rex1 Dependent, but Activation of AKT is not

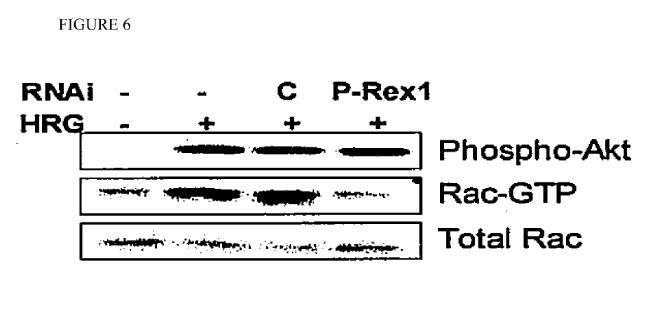

Experiments were also performed to determine whether Akt phosphorylation by HRG in T-47D cells was inhibited by introduction of a P-Rex1 RNAi construct. First, T-47D cells were transfected with RNAi constructs targeting P-Rex1 (SMARTpool, Dharmacon) and incubated for 48 hours. Then, cells were serum-starved and treated with HRG (10 ng/mL) for 5 minutes. Using the PBD pull-down assay and Western blotting, Rac-GTP levels were determined. In addition, an anti-phospho Akt antibody was used in a Western blot. As shown in FIG. 6, depleting expression of P-Rex1 protein by RNAi did not inhibit Akt phosphorylation, whereas depleting expression of P-Rex1 protein by RNAi did inhibit activation of Rac.

### Example 6

### P-Rex2, Unlike P-Rex1, is not Expressed in Breast Cancer Cells

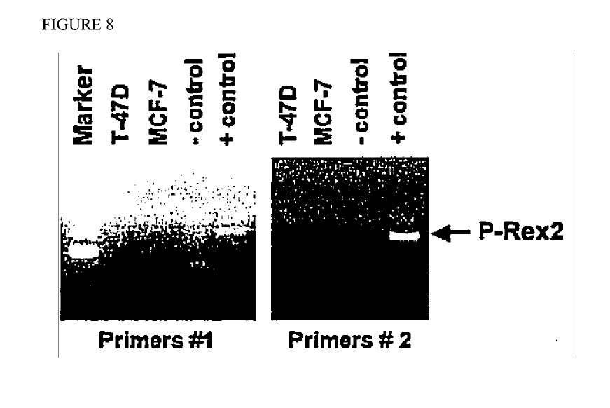

P-Rex2, also called DEPDC2, is a related P-Rex isoform. To determine whether P-Rex2 is expressed in breast cancer cell lines, two sets of primers were designed and implemented in an RT-PCR assay of mRNA isolated from MCF-7 and T-47D cells. No P-Rex2 mRNA was detected (FIG. 8), whereas both sets of primers were able to detect a plasmid construct containing the P-Rex2 gene. This is consistent with the lack of expression of P-Rex2 (DEPDC2) as measured by the Rac-GEF array.

### Example 7

### P-Rex1 is Upregulated in Breast Cancer Cell Lines and Human Breast Tumors as Measured by Quantitative PCR and Immunohistochemistry

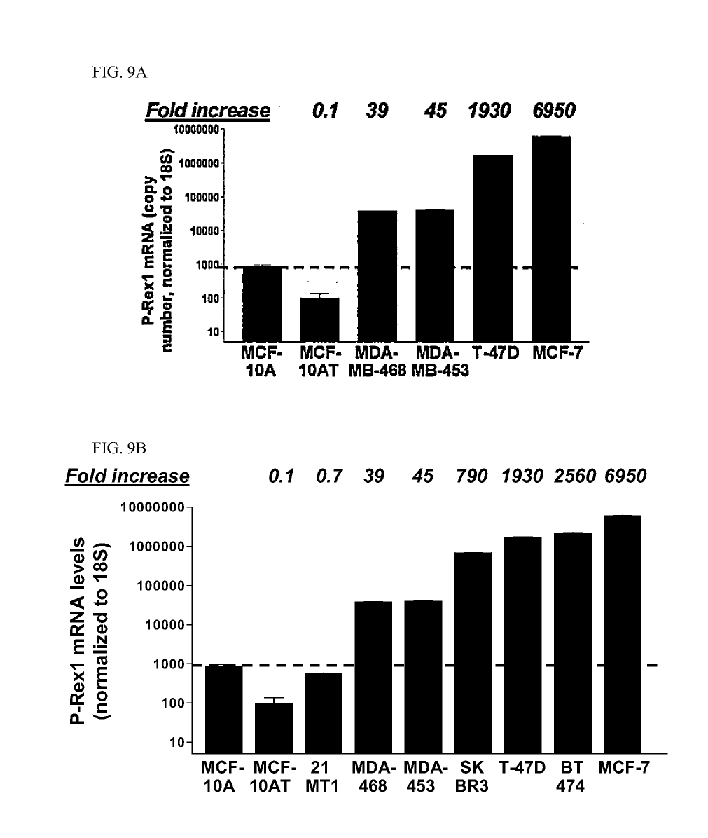

First, the relative expression levels of P-Rex1 mRNA in different breast cancer cell lines were determined; MCF-7, T-47D, MDA-MB-435, MDA-MB-468, MCF-10AT, and MCF-10A cell lines were examined. MCF-7 cells had the highest P-Rex1 expression, followed by T-47D cells (FIG. 9A). In contrast, MDA-MB-453 and MDA-MB-468 had markedly less expression of P-Rex1 relative to that in MCF-7 or T-47D cells. MCF-10A, mammary epithelial cells that, other than being an immortalized cells line, exhibit a non-cancer phenotype, had an even lower expression of P-Rex1 mRNA. Finally, cultured MCF-10AT cells, of human, breast epithelial cell origin that are pre-malignant and proliferate into cancer when grown as xenografts in immune-incompetent mice, also exhibited a very low level of expression of P-Rex1 mRNA. Immunohistochemistry experiments confirmed that the levels of P-Rex1 protein correlated with mRNA levels: Staining with the antibody of Example 1 detected P-Rex1 in MCF-7 and T-47D cells and produced a strong signal, where the same staining of MDA-MB-435 and MDA-MB-468 cells produced a faint/barely detectable signal, and staining of MCF-10A and MCF-10AT cells showed no signal (data not shown).

Overlapping data to that shown in FIG. 9A is shown in FIG. 9B. However, FIG. 9B provides additional data showing the relative expression of mRNA of P-Rex1 in BT-474 cells. Significantly, BT-474 cells normally express very high P-Rex1 levels, overexpress ErbB2/HER2, a common genetic alteration in breast cancer, and depend on ErbB2/HER2 for their ability to develop tumors in nude mice.

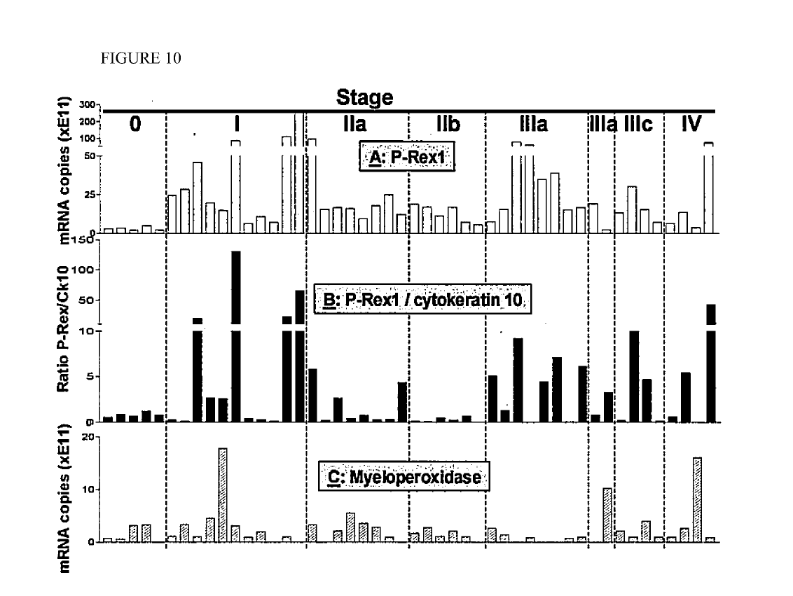

The expression of P-Rex1 in human breast tumors of various stages was also studied, using quantitative PCR of a human tumor cDNA panel (Origene). P-Rex1 was expressed in a number of tumors, but not in non-tumor, i.e. normal, tissue. (FIG. 10, top graph). To ensure that P-Rex1 expression was not simply due to differential representation of epithelial cells in the mixed cell tumor samples, P-Rex1 was normalized to cytokeratin 10 (FIG. 10, middle graph). To ensure that P-Rex1 expression was not due to neutrophil infiltration of the mixed cell tumor samples, where neutrophils are known in the art to express P-Rex1, myeloperoxidase levels were assessed. Myeloperoxidase levels did not correlate with P-Rex1 expression. For example, in some tumors with relatively high P-Rex1 expression, myeloperoxidase levels were very low or not detected. (FIG. 10, bottom graph).

Human breast tumor samples (Dr. Klein-Szanto, Fox-Chase Cancer Center Tissue Bank) were also subject to immunohistochemistry with the antibody of Example 1. Briefly, selected normal and tumor human breast tissues fixed in 10% phosphate-buffered formaldehyde embedded in paraffin were used to evaluate the localization of p-Rex1. Antigen retrieval was accomplished by heating (near boiling) deparaffinized five micron-thick paraffin sections for ten minutes in citrate buffer (pH=6) using a 750 Watt microwave oven at low setting. After pre-incubation in goat serum and peroxidase blocking, the sections were incubated overnight at 4° C. with the a 1:50 dilution of the antibody. Negative controls were incubated overnight in phosphate buffered saline (PBS). After washing with PBS for ten minutes the immunohistochemical reaction was accomplished using a commercial avidin-biotin-peroxidase kit (Vectastain Elite, Vector, Burlingame, Calif.) with diaminobenzidine as chromogen.

FIG. 11 shows two tumor samples that stained positive for P-Rex1 with an antibody derived from a rabbit presented with a peptide consisting of amino acid sequence of SEQ ID NO:1. In initial studies, four of eight tumors positively stained with that antibody, indicating expression of P-Rex1. Furthermore, two samples exhibited particularly strong staining. Staining was only observed in the tumor cells, as indicated by the arrows, and not in the normal ducts or areas surrounding the tumors. Further studies of over 200 samples confirmed this specific staining by the antibody as well as the upregulation of P-Rex1 in human tumor samples. Exemplary results are shown in FIG. 11A.

A table summarizing the results of the staining of the 200 samples is shown in FIG. 11B. Using the antibody of Example 1, P-Rex1 protein expression is detected in 53% (18 of 34) of estrogen receptor-negative tumors and 61% (46 of 75) estrogen-receptor positive tumors. Also using the antibody of Example 1, P-Rex1 protein expression is detected in 51% (51 of 91) HER2 negative tumors and 79% (31/39) of HER2 positive (i.e. HER2 overexpressing) tumors.

These tumor characterizations have important diagnostic and treatment implications. While drugs like tamoxifen can sharply reduce the risk of breast cancers whose growth depends on estrogen, it has no effect on cancers that aren't sensitive to the hormone. Significantly, “estrogen-receptor-negative” or “ER-negative” cancers account for about one-third of breast cancers, or some 65,000 a year in the United States. ER-negative breast cancer cells—unlike those in ER-positive tumors—lack estrogen receptors on their surfaces. Estrogen molecules, therefore, have no port of entry to the cell. Identifying the factor or factors driving the growth and spread of ER negative cancer cells is therefore of great therapeutic interest.

The label of HER2 positive indicates that the breast cancer expresses can be treated with Herceptin, in combination with other chemotherapy drugs. HER2 positive cancers are usually aggressive. There still remains a need to identify additional treatments for HER2 positive and HER2 negative breast cancers.

### Example 8

### HRG Treatment Causes Re-Distribution of P-Rex1 to the Cell Periphery

To test whether HRG treatment caused any change in the sub-cellular localization of P-Rex1, MCF-7 breast cancer cells were serum starved for 48 hours and then either treated for 5 minutes with HRG (10 ng/mL) or left untreated. Cells were then fixed and stained with the antibody of Example 1 to detect P-Rex1 and subject to staining with a secondary Cy2-conjugated antibody, and visualized with fluorescence microscopy. In HRG-treated cells, P-Rex1 staining was distributed along the periphery of the cell, whereas in untreated cells, P-Rex1 staining was more diffuse demonstrating that HRG does cause a change in sub-cellular localization; Specifically, HRG translocates endogenous P-Rex1 to the plasma membrane as shown. (FIG. 12A and FIG. 12B).

To test whether this effect of HRG on P-Rex1 localization was dependent on PI3K, MCF-7 breast cancer cells were serum starved for 48 hours, treated for 10 minutes with HRG (10 ng/mL), with or without Wortmannin (1 μM), an anti-ErbB3 blocking antibody (10 μg/mL, 1 hr) or control IgG and stained and visualized (FIG. 12B). The results shown in FIG. 12B demonstrate that the effect of HRG on P-Rex1 localization is dependent on PI3K signaling. In contrast, the Src inhibitor PP2 and an anti-ErbB4 blocking antibody (10 μg/mL, 1 hr) did not have such an effect (data not shown).

### Example 9

### Generation of P-Rex1-Deficient Cell Lines

T-47D breast cancer cells were transfected with two different lentiviruses (L#1 and L#2) encoding P-Rex1 targeting shRNA (Sigma; MISSION shRNA Control Transduction Particles Catalog number: SHC002V; MISSION Lentiviral Transduction Particles Product number: SHVRS-1. Clone ID: TRCN0000044793 (NM_{—}020820.2-366s1c1); 2. Clone ID: TRCN0000044794 (NM_{—}020820.2-1373s1c1); 3. Clone ID: TRCN0000044796 (NM_{—}020820.2-3777s1c 1); 4. Clone ID: TRCN0000044797 (NM_{—}020820.2-4524s1c 1); MISSION shRNA clones are sequence-verified shRNA lentiviral plasmids for gene silencing in mammalian cells (human or mouse). The parental vector (pLKO.1<-puro) allows for transient transfection or stable selection via puromycin resistance. In addition, the plasmids may be used to generate lentiviral transduction particles in packaging cell lines. MISSION shRNA Lentiviral Particles are transduction-ready viral particles for gene silencing in mammalian cells (human or mouse) including dividing, non-dividing, and primary cell types; www.sigma.com)

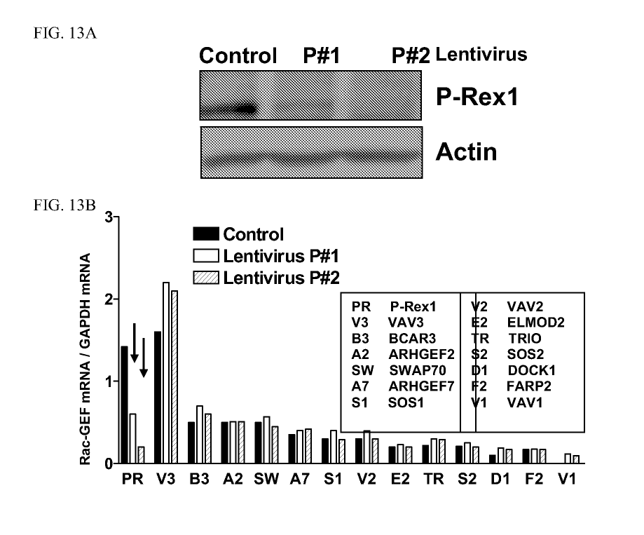

Lentivirus-infected cells were selected with puromycin. Significant P-Rex1 depletion was observed, particularly with L#2 (FIG. 13A). To ensure lentivirus infection and the resulting shRNA expression specifically targeted P-Rex1 and not other Rac-GEFs the Rac-GEF array described in FIG. 3 was developed and used to test expression of Rac-GEFs post-lentivirus infection. Indeed, post-infection depletion was specific for P-Rex1 and not other Rac-GEFs; no significant reduction in mRNA levels of other Rac-GEFs was found (FIG. 13B).

### Example 10

### Breast Cancer Cells with Stably Depleted P-Rex1 Exhibit Defects in Migration and Cell Growth

HRG is known to cause morphological changes, including the formation of lamellipodia and membrane ruffles. Consistent with the Rac activation, when T-47D cells were treated with either HRG or EGF, characteristic ruffles were observed Inhibition of P-Rex1 expression via RNAi resulted in defects in migration, a key step in metastatic dissemination of breast cancer cells and breast cancer development Inhibition of P-Rex1 expression via RNAi also resulted in defects in cell growth in these breast cancer cells.

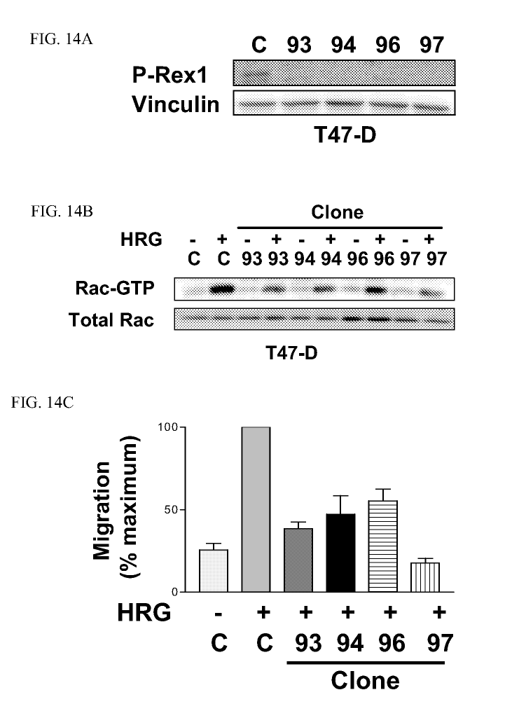

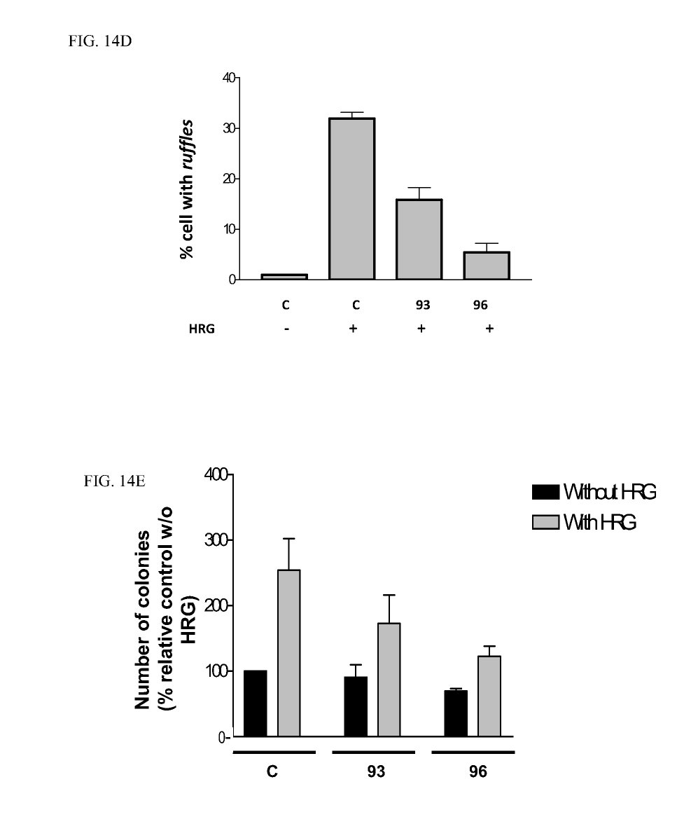

The immunoblot of FIG. 14A depicts the lack of P-Rex1 protein expression in T-47D cells transfected with 4 different pools (93, 94, 96, 97) of lentiviruses that each encode shRNA targeting P-Rex1 (Sigma) and selected using puromycin. FIG. 14B is an image of an immunoblot depicting Rac activation, in response to treatment with HRG (10 ng/mL), in the P-Rex 1 depleted cell lines of FIG. 14A demonstrating ruffles. FIG. 14C is a bar graph plotting the percent migration, in response to treatment with HRG (10 ng/mL), of the P-Rex1 depleted cell lines of FIG. 14A, demonstrating that migration is hindered in P-Rex1 depleted cells lines as measured in a Boyden chamber. FIG. 14D is a bar graph depicting the percentage of cells, wherein the cells are the P-Rex1 depleted cell lines of FIG. 14A, exhibiting decreased ruffling in response to treatment with HRG (10 ng/mL) as compared to control cells. FIG. 14E is a bar graph depicting reduced colony formation, in response to treatment with HRG (10 ng/mL), in the P-Rex1 depleted cell lines of FIG. 14A as compared to control cells.

### Example 10

### Pertussis Toxin (PTX) Partially Inhibits Rac Activation by HRG and EGF Involvement of Gβγ subunits

P-Rex1 is a PI3K- and Gβγ-regulated Rac-GEF. In neutrophils, full activation of this Rac-GEF is achieved by PIP3, the PI3K product, and Gβγ subunits released upon activation of Gi-coupled receptors. It is known that Gβγ alone only causes partial activation of P-Rex1. It is also known that EGF responses, including mitogenesis, are substantially reduced in Pertussis toxin (PTX)-treated cells in many studies. The activation of Gi by EGF and other tyrosine kinase receptors has been validated by a number of laboratories.

As Rac1 activation in MCF-7 and T-47D cells has been shown to be mediated by P-Rex1, whether inhibition of Gβγ release from Gi by PTX inhibited Rac1 activation by HRG was tested. Indeed, in both PTX-treated T-47D and MCF-7 cells, the elevation of Rac1-GTP levels by HRG is markedly impaired. A representative experiment is presented in FIG. 15A. The effect of PTX is consistently partial (˜50-60% inhibition) in both T-47D and MCF-7 cells, as revealed by densitometric analysis (FIG. 15B). In addition, PTX inhibited the activation of Rac by EGF (FIG. 15C).

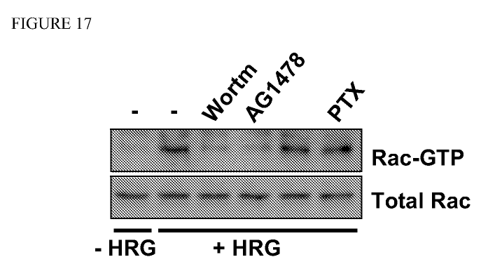

In contrast, the data in FIG. 17 demonstrate that Rac1 activation in non-transformed but immortalized breast cells of line MCF-10A is insensitive to PTX. The image of the immunoblot depicts Rac1-GTP levels as determined by a GST pull down assay of serum-starved MCF-10A cells treated with HRG (100 ng/ml, 5 min), with or without pretreatment with PI3K inhibitor wortmannin, EGFR inhibitor AG1478, PI3Kγ inhibitor 5-quinoxalin-6-ylmethylene-thiazolidine-2,4-dione (1 mM, 1 h), or PTX (24 h) (FIG. 17). Two additional experiments gave similar results.

Taken together, these results indicate the involvement of Gβγ subunits in the activation of Rac1 by ErbB receptors, and provide support for that the signaling mechanism is trans-activation via a Gi-coupled receptor.

Furthermore, the involvement of multiple receptors is supported (data not shown). In fact, P-Rex1 also mediates Rac1 activation by EGFR and ErbB2 overexpression. Since it was found that activation of Rac1 and Rac responses induced by HRG are dependent on the EGFR, whether P-Rex1 depletion could also affect direct activation of Rac1 by ErbB1 ligands was examined. T-47D cells were subject to P-Rex1 RNAi (SMARTpool®RNAi, Dharmacon©; ON-TARGET Plus© Non-targeting Pool D-001810-10-05; ON-TARGET plus SMART pool L-010063-01-0005, human PREX1, NM_{—}020820; SMARTpoo10 reagents feature four different siRNA duplexes, which reduces off-target effects. Each siRNA has a unique off-target signature, which can be identified by microarray analysis. When the siRNA is present as a fraction of the total concentration as with a SMARTpoo10 reagent, Dharmacon (Fisher Scientific) has found that the off-target effects from any one siRNA are minimized and reduced. Any sequence-independent effects of the siRNAs will either be the same as with a single duplex or reduced. The nucleic acid constructs feature proprietary chemical modifications; Duplexes: 1. J-010063-09, 2. J-010063-10, 3. J-010063-11; 4. J-010063-12; www.dharmacon.com).

After 48 h cells were serum-starved, treated with EGF or TGF-α(10 ng/ml, 2 min) and Rac-GTP levels were determined using a PBD pull-down assay. T-47D cell clones (control and P-Rex1-depleted, clone L#2) were transfected with the ErbB 1 mammalian expression vector pcDNA-ErbB1. After 48 h, cells were serum-starved and Rac-GTP levels were determined. A marked inhibition of both EGF- and TGF-α-induced Rac1 activation in P-Rex1 knock-down T-47D cells was found (data not shown), suggesting that inputs from multiple ErbB receptors can signal to Rac1 activation via P-Rex1.

It is known that ErbB2 overexpression leads to Rac activation both in breast cancer cellular and in vivo models. To evaluate the potential involvement of P-Rex1 in this context, ErbB2 was expressed in T-47D cells, which normally express low ErbB2 levels. T-47D cells were grown and transfected as with the experiments with ErbB1, but using an ErbB2 expression vector. ErbB2 overexpression led to elevations in Rac-GTP levels in serum-starved T-47D cells (data not shown). On the other hand, when ErbB2 is overexpressed at similar levels in P-Rex1-depleted T-47D cells, Rac1-GTP levels are not elevated (data not shown).

Therefore, P-Rex1 signals to Rac not only in response to ErbB3 and EGFR ligands but also in response to ErbB2 overexpression. In all, this suggests that P-Rex1 is a common mediator of Rac activation downstream of multiple ErbB receptors.

### Example 11

### PI3Kγ Mediates HRG Activation of Rac1

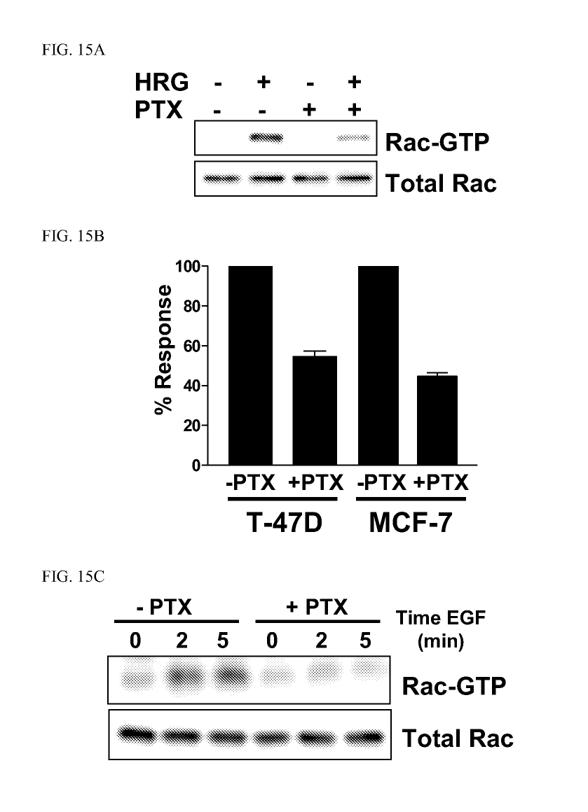

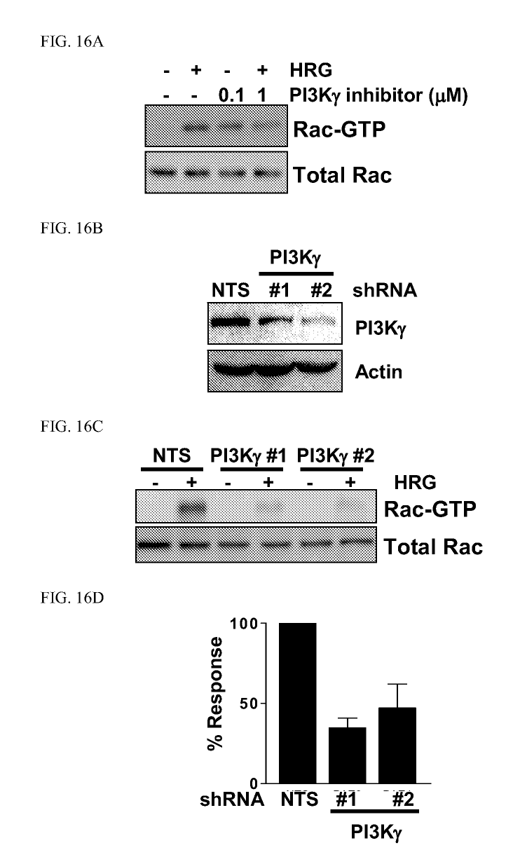

FIG. 16 demonstrates that P-Rex1 is translocated to the cell periphery (membrane ruffles) in response to heregulin (HRG) in a PI3K-dependent manner.

As described above, Gβγ subunits are known to activate PI3Kγ. As Rac activation by HRG is affected by PTX as shown in Example 10, the involvement of PI3Kγ in the response was investigated. Thus, the pharmacological PI3Kγ inhibitor 5-quinoxalin-6-ylmethylene-thiazolidine-2,4-dione was tested; it dose-dependently reduced Rac activation by HRG in MCF-7 cells (100 ng/ml, 5 min) (FIG. 16A). Similar results were shown in T-47D cells (data not shown).

To further investigate the role of PI3Kγ, PI3Kγ depleted MCF-7 cell lines were generated using PI3Kγ (#1 and #2) and non-target sequence (NTS) control shRNA lentiviruses (Sigma) (FIG. 16B). A significant reduction in Rac1 activation by HRG was observed in the cells expressing shRNA targeting PI3Kγ as compared to cells infected with a control (non-targeting sequence) shRNA lentivirus (FIG. 16C).

Densitometric analysis of the cells in FIG. 16B and FIG. 16C revealed an inhibition of approximately 60% upon PI3Kγ depletion by RNAi, as compared to the control non-target sequence (FIG. 16D). Therefore, PI3Kγ is implicated in Rac1 activation downstream of ErbB receptors, at least in response to HRG.

The data in FIG. 17 demonstrate that Rac1 activation in non-transformed but immortalized breast cells of line MCF-10A is insensitive to PI3Kγ inhibitor but is sensitive to wortmanin. The image of the immunoblot depicts Rac1-GTP levels as determined by a GST pull down assay of serum-starved MCF-10A cells treated with HRG (100 ng/ml, 5 min), with or without pretreatment with PI3K inhibitor wortmannin, EGFR inhibitor AG1478, PI3Kγ inhibitor 5-quinoxalin-6-ylmethylene-thiazolidine-2,4-dione (1 mM, 1 h), or PTX (24 h) (FIG. 17). Two additional experiments gave similar results.

The fact that Rac activation in MCF-10A cells is wortmannin-sensitive suggests that it is mediated by a PI3K-sensitive Rac-GEF. However, neither the PI3Kγ inhibitor nor pretreatment with PTX reduced Rac activation by HRG. These results are very important because they suggest that stimulation of ErbB3 transduces signals to Rac independently of P-Rex1 when this Rac-GEF is not expressed. The EGFR inhibitor AG1478 also blocked Rac activation, suggesting that that ErbB receptor cross-talk operates in these cells, although Rac activation may have a different utilization of Rac-GEFs than that in P-Rex1 overexpressing cells such as MCF-7 or T-47D cells. Interestingly, experiments using the specifically designed Rac-GEF array, which is described above, showed the highest expression for Vav3, Sos1, and Trio in MCF-10A cells, whereas P-Rex1 mRNA levels were almost undetectable (data not shown).

### Example 13

### CXCR4 Mediates HRG-Induced Activation of Rac1

Cross-talk between tyrosine kinase receptors and GPCRs, including receptor transactivation in both directions, has been extensively studied in a variety of signaling network experiments. Interestingly, numerous studies showed that Gi-coupled-receptors are involved in growth factor receptor-mediated responses, including those triggered by EGF, PDGF and NGF For example, it is known that PDGF-induced migration is dependent upon the activation of the GPCR EDG-1. Most recently, studies in MDA-MB-435 cells (originally thought to be a breast cancer cell line and then reclassified as a melanoma cell line) showed that the metastatic response driven by ErbB2 is mediated by CXCR4, a Gi-coupled receptor for the chemokine stromal cell-derived factor 1 (SDF-1/SDF-1α). SDF-1 is highly expressed and released from both cancer and stromal cells, including in breast cancer, and CXCR4 is highly expressed in human metastatic breast tumors. CXCR4 activation drives proliferation, migration, and invasion of breast cancer cells. Estradiol also promotes T-47D breast cancer cell proliferation by enhancing SDF-1α release and EGFR transactivation.

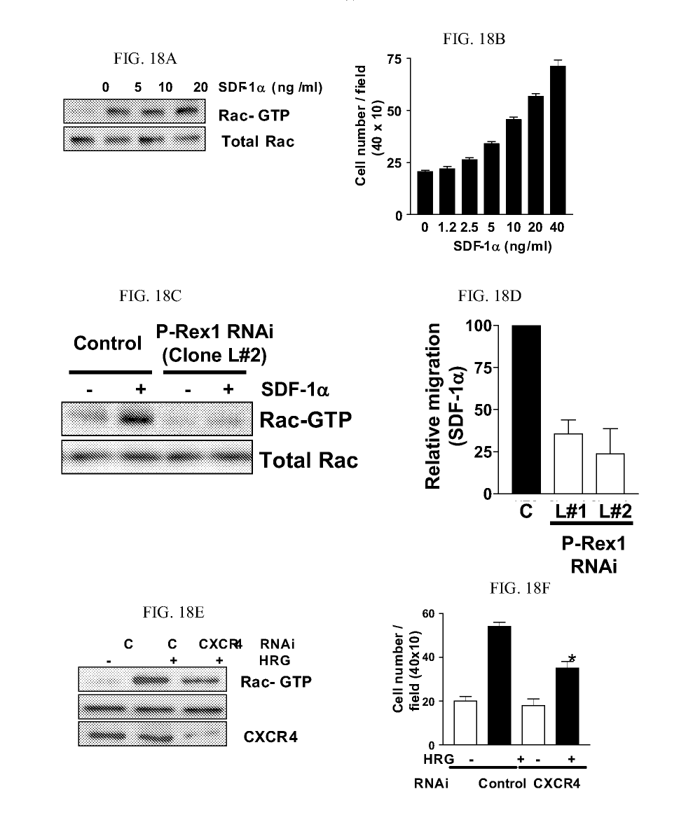

Since CXCR4 had the ability to activate Rac1 via P-Rex1 in breast cancer cells, the ability of SDF-1a to do so was tested. Indeed, in MCF-7 cells, SDF-1a strongly activates Rac1 (FIG. 18A) and induces a migratory response, as assessed with a Boyden chamber assay (FIG. 18B).

Similar results were observed in T-47D cells (data not shown). Rac1 activation and migration induced by SDF-1a are markedly impaired in P-Rex-1-deficient T-47D cells (FIGS. 18A and 18B). Thus SDF-1α-induced activation of Rac1 in these cells is mediated by P-Rex1.

To determine whether CXCR4 is involved in HRG responses, CXCR4 expression was knocked down using RNAi in MCF-7 cells. Remarkably, Rac1 activation was significantly reduced in CXCR4-depleted MCF-7 cells (61±3% inhibition, n=3) (FIG. 18E). Motility in response to HRG was also dependent on CXCR4 (FIG. 18F). Similar results were observed with a second RNAi duplex (data not shown). Altogether, these results argue for a transactivation mechanism by which ErbB receptors via CXCR4 mediate P-Rex1 and Rac1 activation. Such a mechanism has important implications in tumorigenesis and metastatic dissemination.

### Example 14

### Signaling Schematic

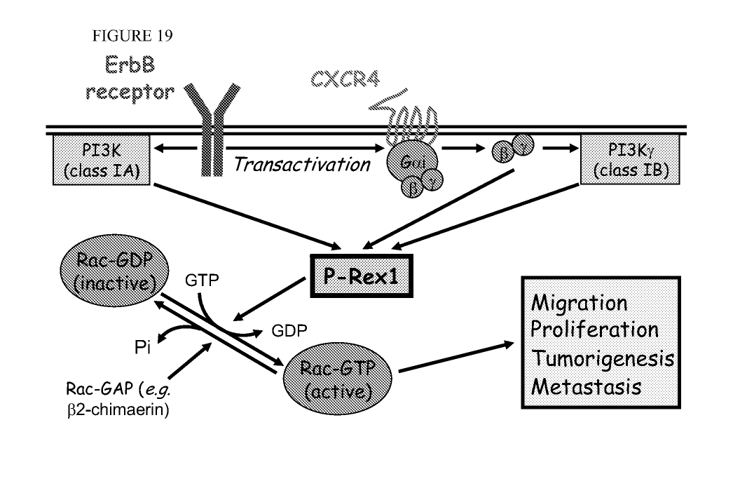

In sum, the data above represent significant implications for HRG and EGF signaling in breast cancer. First, overexpression of ErbB2 in MCF-7 cells elevates Rac-GTP levels, and that more importantly, the effect is lost in P-Rex1-depleted cells. Second, pertussis toxin (PTX), which inhibits the dissociation of Gi, substantially reduces Rac activation by HRG and EGF in MCF-7 and T-47D cells (FIG. 15). Third, pharmacological inhibition and RNAi depletion of PI3Kγ, a direct downstream target of Gβγ subunits, also impairs Rac activation by HRG in MCF-7 cells (FIG. 16). Fourth, in MCF-10A cells, which do not express detectable P-Rex1, activation of Rac by HRG is insensitive to PTX and a PI3Kγ inhibitor (FIG. 17); thus, the Gβγ and PI3Kγ requirement can be recapitulated only in those cell lines that express high P-Rex1, such as MCF-7 or T-47D cells. Fifth, it is demonstrated that CXCR4, a Gi-coupled receptor, is implicated in HRG-induced Rac1 activation. CXCR4-depleted MCF-7 cells demonstrated impaired Rac1 activation by HRG. Moreover, in breast cancer cells the CXCR4 ligand, SDF-1a, activates Rac1 and induced motility via P-Rex1 (FIG. 18). This latter result is highly relevant because CXCR4 has been widely implicated in the progression of several cancers, including breast cancer, and its expression correlates with poor prognosis. A study in breast cancer cells reported that CXCR4 and Gi exist as a complex together with the tyrosine kinase receptor IGF-RI. IGF causes the dissociation of the complex and Gβγ release. Moreover IGF-induced migration is PTX-sensitive. A similar scenario is likely for ErbB receptors. Upon activation CXCR4 releases Gβγ subunits, and in addition to its direct effect on P-Rex1, Gβγ subunits activate class IB PI3Ks (PI3Kγ), which may ultimately contribute to P-Rex1 activation. Thus, P-Rex1 receives inputs from class Ia PI3Ks (which are activated by ErbB receptors) and class Ib PI3K (activated by Gβγ). Notably, PI3Kγ is implicated in oncogenesis and invasion. Together, these results suggest a signaling network shown in FIG. 19.

### Example 15

### P-Rex1 Depletion Impairs HRG-Mediated Tumorigenicity In Vivo in Nude Mice

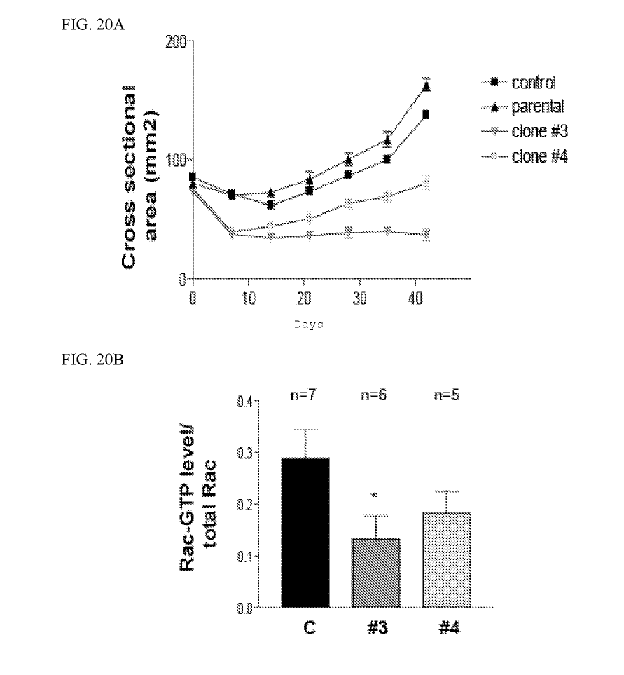

The results shown in FIGS. 20A and 20B demonstrate that HRG mediated tumor formation can be inhibited in vivo by depleting P-Rex1 expression

For the stable knockdown of P-Rex1, BT-474 cells were infected with P-Rex1 shRNA mission lentiviral transduction particles (Sigma) according to manufacturer's protocol and selected with puromycin 1 ug/ul. Briefly, cells were seeded at 80% confluence and on the following day infected with 1 MOI of P-Rex1 lentivirus and left in these conditions for three days. Subsequently, 1 ug/ul puromycin was added into the media and cells were cultured for 2 weeks to select for a multiclonal pool. Successful knockdown of P-Rex1 expression was tested by western blot and RT-PCR (data not shown).

P-Rex1-depleted cell lines or control cells were injected into the flanks of athymic (nude) female ovariectomized mice (2×10⁷ cells/mouse). The mice (10 animals per cell line) were checked daily for tumor formation by palpation, and after tumors were detected, tumor cross sectional area was measured once a week. The mouse experiments were conducted under an approved animal protocol. The results are shown in FIG. 20A, wherein the graph plotting tumor area versus number of days shows inhibition of tumor formation with of P-Rex1-depleted BT-474 cell lines as compared to control cells. The parental cell line and the cells treated with a control nucleic acid construct form tumors, while 2 cell lines in which P-Rex1 was depleted using 2 different RNAis (clones #3 and #4) do not form tumors and/or or exhibiting a significant loss of tumorigenic potential.

Decreased levels of Rac activation were also observed in vivo in the P-Rex1 depleted tumor cell lines. The results are shown in FIG. 20B, wherein the bar graph depicts decreased levels of Rac activation, as compared to control, in the P-Rex1 depleted BT-474 cell lines used in the experiment shown in FIG. 20A.

This demonstrates that inhibiting P-Rex1 expression or activity inhibits tumorigenesis in vivo. Significantly, the BT-474 cells express very high P-Rex1 levels, overexpresses ErbB2/HER2, a common genetic alteration in breast cancer, and depend on ErbB2/HER2 for their ability to develop tumors in nude mice.

1. A method of detecting cancer in a sample of mammalian breast tissue comprising: measuring the level of expression of P-Rex1 in the sample and comparing that level with the level of expression of P-Rex1 in a control sample.

2. The method of claim 1 wherein the mammal is a human.

3. The method of claim 2 wherein the comparing step is of the levels of expression of P-Rex1 mRNA.

4. The method of claim 3 wherein the levels of expression of P-Rex1 mRNA are measured by quantitative RT-PCR.

5. The method of claim 1 wherein the comparing step is of the levels of expression of P-Rex1 protein.

6. The method of claim 5 wherein expression of P-Rex1 protein is measured using an antibody to P-Rex1.

7. The method of claim 5 wherein expression of P-Rex1 protein is measured using an antibody to P-Rex1, wherein the antibody is derived from an animal presented with a peptide comprising the amino acid sequence of SEQ ID NO:1.

8. An antibody to P-Rex1 protein derived from an animal presented with a peptide comprising the amino acid sequence of SEQ ID NO:1.

9. An antibody to P-Rex1 protein derived from an animal presented with a peptide consisting of the amino acid sequence of SEQ ID NO:1.

10. An antibody of claim 8 or 9 in humanized form.

11. A method of determining P-Rex1 expression in a sample comprising contacting the sample with an antibody derived from an animal which has been presented with a peptide comprising the amino acid sequence of SEQ ID NO:1 and detecting binding of the antibody with at least some, if any, P-Rex1 present in the sample.

12. A method of determining P-Rex1 expression in a sample comprising contacting the sample with an antibody derived from an animal which has been presented with a peptide consisting of the amino acid sequence of SEQ ID NO:1 and detecting binding of the antibody with at least some, if any, P-Rex1 present in the sample.

13. A method of identifying a compound useful for treating breast cancer comprising contacting the compound with breast cancer cells and comparing the expression or activity of P-Rex1 in the cells contacted with the compound with the expression or activity of P-Rex1 in similar cells not contacted with the compound, wherein a compound that reduces the level of expression or activity of P-Rex1 is determined to have such utility.

14. The method of claim 13 wherein the compound is a nucleic acid construct.

15. The method of claim 13 wherein the compound is a nucleic acid comprising an shRNA, siRNA, or antisense construct, or a combination thereof.

16. A method of inhibiting breast cancer progression comprising inhibiting P-Rex1 activity or expression, or both.

17. The method of claim 16 wherein inhibiting breast cancer progression comprises inhibiting breast cancer metastasis by inhibiting P-Rex1 activity or expression, or both.

18. A method of treating breast cancer comprising inhibiting P-Rex1 activity or expression, or both.

19. The method of any one of claims 17-18 wherein inhibiting P-Rex1 activity or expression, or both, comprises administering a nucleic acid comprising an shRNA, siRNA, or antisense construct, or a combination thereof.

20. The method of any one of claims 13-18 wherein the breast cancer is estrogen receptor negative or HER2 positive, or both.

21. The method of claim 19 wherein the breast cancer is estrogen receptor negative or HER2 positive, or both.


SEQUENCE LISTING (1 sequence)

SEQ ID NO: 1 - protein, 22 aa, Artificial Sequence
  Synthetic construct
QEEDQADSAFPLLSLGPRLSLC